(19) United States
(12) Patent Application Publication
(10) Pub. No.: US 2022/0282295 A1
(43) Pub. Date: Sep. 8, 2022

(54) SINGLE-CELL LEVEL CONNECTOMICS USING A DNA-SYNTHESIS BASED BARCODING SYSTEM AND METHODS OF USING THE SAME

(71) Applicant: Northwestern University, Evanston, IL (US)

(72) Inventor: Keith E.J. Tyo, Evanston, IL (US)

(73) Assignee: Northwestern University, Evanston, IL (US)

(21) Appl. No.: 17/687,276

(22) Filed: Mar. 4, 2022

Related U.S. Application Data
(60) Provisional application No. 63/157,521, filed on Mar. 5, 2021.

(51) Int. Cl. C12P 19/34; C12N 9/12; C12Q 1/6869

(52) U.S. Cl. CPC C12P 19/34; C12N 9/1264; C12Q 1/6869; C12Y 207/07031

(57) ABSTRACT

The present disclosure provides a DNA-synthesis based recording system that, in combination with CRISPR-Cas9 or other CRISPR systems, can establish single-cell level connectivity for densely packed cells, for example in the brain.

### CROSS-REFERENCE TO RELATED APPLICATIONS

This application claims priority to U.S. Provisional Application No. 63/157,521, filed Mar. 5, 2021.

### STATEMENT REGARDING FEDERALLY SPONSORED RESEARCH OR DEVELOPMENT

This invention was made with government support under grant number NS107697 awarded by National Institutes of Health (NIH). The government has certain rights in the invention.

### BACKGROUND

The following discussion is provided to aid the reader in understanding the disclosure and is not admitted to describe or constitute prior art thereto.

Single cell connectomics can reveal extremely relevant biological information regarding tumor growth, neuronal connections, organoid growth, developmental biology¹⁻⁴ etc. Several techniques have been developed for lineage tracing⁵⁻⁷ and connectomics^{8,9} however most of them lack single cell resolution. This is either due physical limitations of optical or electrical probes^{10,11} or the limited recording characteristics of DNA-based devices^{12,13}. The present disclosure provides systems and methods for overcoming the limitations of prior systems.

### SUMMARY

The present disclosure provides DNA-synthesis based recording systems that, in combination with CRISPR-Cas9 or other CRISPR systems, can establish single-cell level connectivity for densely packed cells such as those within the brain, for example.

In one aspect, the present disclosure provides DNA-synthesis based recording systems, comprising a Cas, a homing guide RNA (hgRNA), and a terminal deoxynucleotidyl transferase (TdT), wherein the Cas, the hgRNA, and the TdT are all comprised within a single cell.

The Cas can be Cas9, or other known Cas proteins (e.g., Cas3, Cas4, Cas8a, Cas5, Cas8b, Cas8c, Cas10, Cas12, Cas13).

In some embodiments, the Cas (e.g., Cas9) forms a complex with hgRNA and targets a DNA locus of the hgRNA.

In some embodiments, the hgRNA spacer sequence is diversified after each edit.

In some embodiments, the TdT is directed to the double-stranded breaks created by Cas at the hgRNA sites, and the TdT adds at least one nucleotide at the double-stranded breaks, and wherein the at least one nucleotide optionally comprises a barcode. In some embodiments, the identity of the nucleotide added by TdT depends on the concentration of nucleotides in the cell. In some embodiments, the TdT-directed base additions can be altered by altering the nucleotide concentration.

In some embodiments, a change in TdT-based nucleotide incorporation into a hgRNA double-stranded break is defined as an output signal. In some embodiments, the output signal is detectable with in situ sequencing.

In some embodiments, the cell is a neuron. In some embodiments, the neuron is within the brain of a living mammal.

In another aspect, the present disclosure provides methods of establishing connections between cells, comprising exposing at least two cells that each comprise a DNA-synthesis based recording system according to claim 1 to an organic environment comprising deoxyribonucleotide triphosphates (dNTPs) and a variable, allowing the TdT to add dNTPs to a DNA substrate, and isolating the DNA substrate; wherein the dNTP content of the DNA substrate corresponds to the concentration of the variable in the organic environment.

The foregoing general description and following detailed description are exemplary and explanatory and are intended to provide further explanation of the invention.

### BRIEF DESCRIPTION OF THE DRAWINGS

FIG. 1A shows a conceptual presentation of homing guide RNA system.

FIG. 1B shows a conceptual representation of TdT-directed nucleotide additions in the homing guide RNA locations.

FIG. 1C shows a conceptual representation of establishing a mouse model for tracing a single cell level spatial connectivity in mouse brains using our proposed TdT-based recording system.

FIG. 2A shows the output signal for each nucleotide upon treating HEK 293T cells with the listed concentrations of dAdo.

FIG. 2B shows the output signals for each nucleotide upon treating HEK 293T cells with the listed concentrations of dThd.

FIG. 2C shows the output signals for each nucleotide upon treating HEK 293T cells with the listed concentrations of dGuo.

FIG. 2D shows the expected output signal for various NTP incorporations in a tissue sample based on the distance of the cells from the site of injection of input signal.

### DETAILED DESCRIPTION

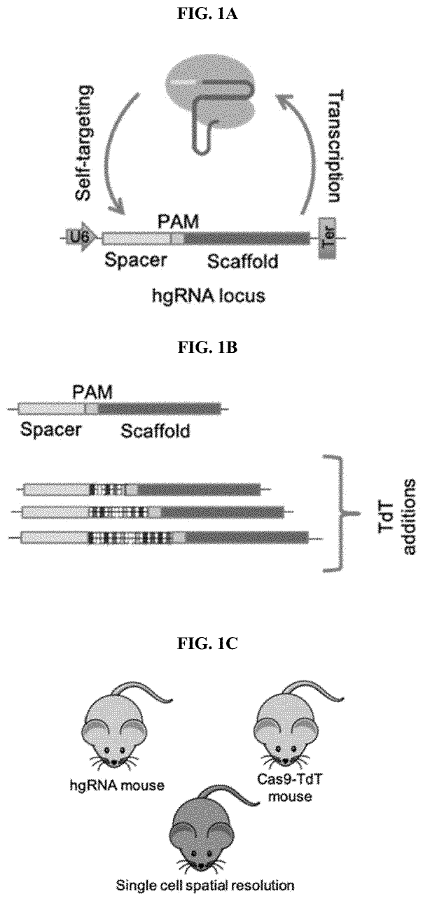

The present disclosure provides a DNA-synthesis based recording system that, in combination with CRISPR-Cas9 or other CRISPR systems, can establish single-cell level connectivity for densely packed cells, for example in the brain. The disclosed system has been prototyped in HEK-293T cells (data included here) and can be adapted to mammalian brains, such as a mouse brain. A special version of guide RNA scaffold was used, called the homing guide RNA (hgRNA), in which a protospacer adjacent motif (PAM) sequence is added after the spacer, such that the Cas9-hgRNA complex targets the DNA locus of the hgRNA itself (FIG. 1A). This leads to diversification of the hgRNA spacer sequence after each edit. By co-expressing terminal deoxynucleotidyl transferase (TdT) in this system, the TdT is directed to the double-stranded breaks created by Cas9 (or related Cas derivatives and analogs) at the hgRNA sites and add nucleotides in HEK 293T cells (FIG. 1B).

The identity of these nucleotides added by TdT depends on the concentration of nucleotides in the cell, and the TdT-directed base additions can be altered by altering the intracellular nucleotide concentration. One of the ways the nucleotide pool can be altered is by dosing the cell culture media with deoxyribonucleosides, such as deoxyadenosine (dAdo), deoxyguanosine (dGuo), deoxcytosine (dCyt), and deoxythymidine (dThd), since these are readily taken up by cells.

Optionally, cells used for the disclosed systems and methods may be treated with an adenosine deaminase inhibitor, such as pentostatin (deoxycoformycin, dCF), which inhibits degradation of dAdo that would otherwise result in an altered nucleotide pool.

Added small molecules (e.g., dAdo, dGuo, dCF etc) can be utilized as an input signal. The percentage change in the TdT-based nucleotides incorporated into the hgRNA sites can be utilized as an output signal. For example, the input signal may be the overall concentration or individual concentration of the TdT-based nucleotides, and the output signal is the percent of those TdT-based nucleotides found in the DNA locus of the hgRNA.

Further, this system allowed for the recording of a dose response in the output signal for varying concentrations of the input signals of dAdo, dThd and dGuo. Each increasing concentration of TdT-based nucleotide input signal (e.g., 0 to 500 μM) may result in a higher output signal. For example, an increase in dAdo may increase the corresponding output signal. None of the current genetically encoded signal recording systems synthesize a DNA-based record in a dose response manner in mammalian cells. This salient feature of being able to synthesize a record of various NTP concentrations in the genome of cells can help in the spatial reconstruction of tightly packed together cells in tissue. Moreover, since the output signal is high (e.g. with 5 mM dThd), several lower concentrations can be distinguished, thus providing a single-cell level resolution.

Based on the data provided herein, the system may be used for single-cell spatial resolution in mammalian brains or other tissues or organs, as disclosed herein.

For example, in some embodiments, the disclosed systems and methods can be used for molecular recording in vivo, such as in the brain of an animal (e.g., a mammal, such as a mouse, dog, cat pig, sheep, cow, horse, or human). To establish the system in vivo, preassembled Cas9 ribonucleotideprotein (RNP) and TdT can be delivered into the brain of an animal using intracranial injection system. In parallel, a Cas9-TdT fusion, attached by a T2A self-cleaving linker can be expression in primary neurons of the animal via transfection. For both these systems, the percentage of cells in which TdT based edits can be recorded and the percentage of each nucleotide incorporation can be calculated to establish a “0” control condition. “0” can be defined as a no input signal control.

The disclosed methods may comprise sequencing in order to determine the percentage of each nucleotide incorporation into the DNA loci of the hgRNA. In some embodiments, sequencing may comprise next-generation sequencing (NGS), true single molecule sequencing (tSMS), 454 sequencing, SOLiD sequencing, ion torrent sequencing, single molecule real time (SMRT) sequencing, Illumina sequencing, nanopore sequencing, or chemical-sensitive field effect transistor (chemFET) sequencing.

Next-generation sequencing (NGS) methods share the common feature of massively parallel, high-throughput strategies, with the goal of lower costs and higher speeds in comparison to older sequencing methods. NGS methods can be broadly divided into those that require template amplification and those that do not.

Sequencing techniques that find use in some embodiments herein include, for example, Helicos True Single Molecule Sequencing (tSMS) (Harris T. D. et al. (2008) Science 320:106-109). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm′. The flow cell is then loaded into a sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step. Further description of tSMS is shown for example in Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), each of which is incorporated by reference in their entireties.

Another example of a DNA sequencing technique that finds use in some embodiments herein is 454 sequencing (Roche) (Margulies, M et al. 2005, Nature, 437, 376-380; incorporated by reference in its entirety). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments are attached to DNA capture beads, e.g., streptavidin-coated beads using, e.g., Adaptor B, which contains a 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that finds use in some embodiments herein is SOLiD technology (Applied Biosystems). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

Another example of a DNA sequencing technique that finds use in some embodiments herein is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982; incorporated by reference in their entireties). In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and are attached at a resolution such that the fragments are individually resolvable. Addition of one or more nucleotides releases a proton (W), which is detected and recorded in a sequencing instrument. The signal strength is proportional to the number of nucleotides incorporated.

Another example of a DNA sequencing technique that finds use in some embodiments herein is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a DNA sequencing technique that finds use in some embodiments herein is the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a DNA sequencing technique that finds use in some embodiments herein involves nanopore sequencing (Soni G V and Meller A. (2007) Clin Chem 53: 1996-2001; incorporated by reference in its entirety). A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a DNA sequencing technique that finds use in some embodiments herein involves using a chemical-sensitive field effect transistor (chemFET) array to sequence DNA (for example, as described in US Patent Application Publication No. 20090026082; incorporated by reference in its entirety). In one example of the technique, DNA molecules can be placed into reaction chambers, and the template molecules can be hybridized to a sequencing primer bound to a polymerase. Incorporation of one or more nucleoside triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In some embodiments, other sequencing techniques (e.g., NGS techniques) understood in the field, or alternatives or combinations of the above techniques find use in some embodiments herein.

The recording set-up can be repeated with the addition and variation of an input signal, e.g., with direct injection of either deoxyribonucleosides or other small molecules known to alter intracellular nucleotide pools (like dCF etc). This can be done with dAdo, dGuo, dThd, dCyt and dCF. This will establish the condition that gives the maximum output signal in neurons. Finally, establish how the dose response will look for the best signal recorded. Trying at least 10 different concentrations for that input signal. Characterizing the control conditions in detail will be very important for the final application of spatial reconstructions.

The cell/cells/tissue/animal expressing the disclosed recording system can be treated with the RNP and TdT in, with the relevant input signal established above. After recording, a sample from the cell/cells/tissue/animal can be obtained and sequenced. Preserving tissue samples in tissue sections can help supplement the spatial resolution even more. Since the concentration of the input signal will slowly diffuse over the depth of the tissue sample, sequencing the output signal at different locations will help us establish connectivity.

The disclosed systems and methods may suitable for recording in a single cell, a cluster or group of cells, a tissue, and organ, or an entire organism/animal. In some embodiments, recordings may be made concurrently across an entire tissue or organ, such as the brain (e.g., the brain of a mammal).

Because the disclosed recording system is continuous (records by dNTP additions over time, under all the varying input signal conditions) and it is fully genomically encodable, it will allow for coverage of the entire brain, reducing deciphering the connectome to a DNA sequencing problem. While in situ sequencing can be labor intensive, it is possible to, in parallel, link the TdT based additions to single cell barcodes, which can eliminate the need for in situ sequencing. Further, by engineering TdT to respond to input signals like calcium, once optimized such engineered TdT when incorporated into the disclosed system, can further establish a functional connectome at a single cell level. While the disclosed experiments may not provide a large output signal, it is believed that even with a small output signal the connectome can be establish at least at a population level for about 100-1000 neurons per population. Finally, this method and system can be employed to study tumor growth, embryo development, and other developmental processes and cellular connections.

All references disclosed herein are specifically incorporated by reference thereto.

While preferred embodiments have been illustrated and described, it should be understood that changes and modifications can be made therein in accordance with ordinary skill in the art without departing from the invention in its broader aspects as defined herein.

### EXAMPLES

### Example 1

The disclosed system has been prototyped in HEK-293T cells. A hgRNA was co-expressed with terminal deoxynucleotidyl transferase (TdT), and Cas9. The TdT was directed to the double-stranded breaks created by Cas9 at the hgRNA sites and added nucleotides in HEK 293T cells (FIG. 1B).

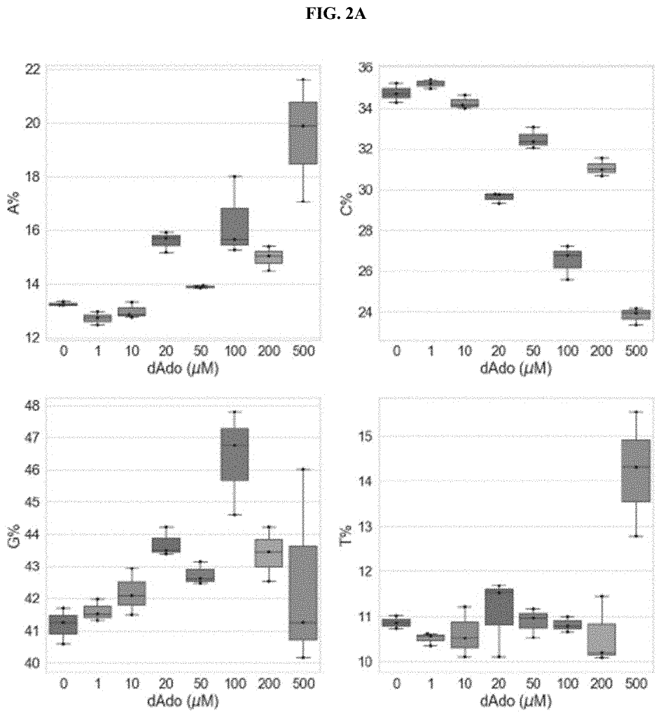

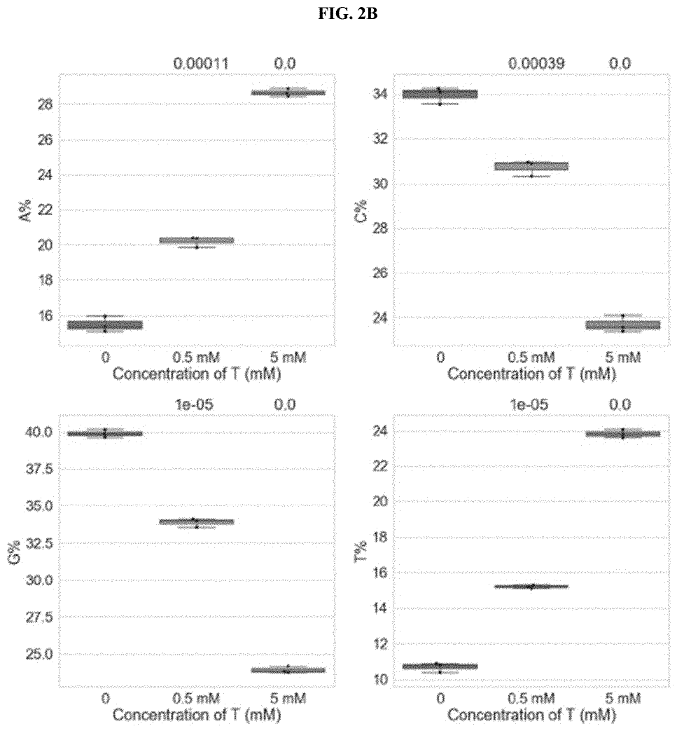

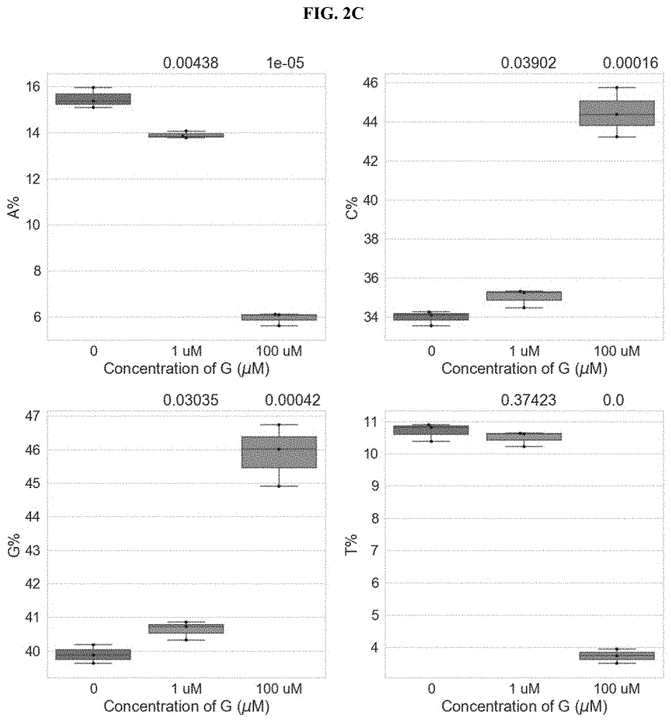

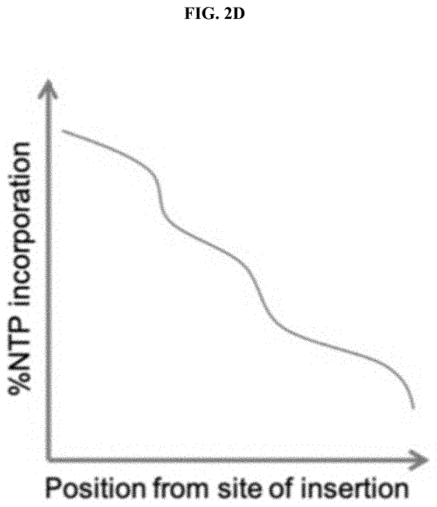

HEK 293T cells were treated with an adenosine deaminase inhibitor, pentostatin (deoxycoformycin, dCF), that inhibits degradation of dAdo, resulting in an altered nucleotide pool. The added small molecules (dAdo, dGuo, dCF etc) were defined as the input signal. The percentage change in the TdT-based nucleotides incorporated into the hgRNA sites was defined as the output signal. For example, for an input signal of 5 mM dThd, an output signal was observed of 8% for A, 10% for C, 17.5% for G and 20% for T (FIG. 2B). This shows a high output signal response for dAdo, dGuo and dCF input signals as well.

Further, this system allowed for the recording of a dose response in the output signal for varying concentrations of the input signals of dAdo, dThd and dGuo (FIGS. 2A, 2B and 2C). Each increasing concentration of dAdo input signal (0 to 500 μM) resulted in a higher output signal (except for 50 μM treatment; 500 μM treatment was toxic HEK cells) (FIG. 2A). None of the current genetically encoded signal recording systems^{12,13} synthesize a DNA-based record in a dose response manner in mammalian cells. This salient feature of being able to synthesize a record of various NTP concentrations in the genome of cells can help in the spatial reconstruction of tightly packed together cells in tissue. Moreover, since the output signal is high (e.g. with 5 mM dThd), several lower concentrations can be distinguished, thus providing a single-cell level resolution.

### Example 2

This prophetic examples explains how the disclosed system can function in a neuronal cell culture and in vivo in mouse brains.

Start with post-mitotic neurons in adult mouse brains and deliver preassembled Cas9 ribonucleotideprotein (RNP) and TdT using intracranial injection system as done previously by Staahl et al¹⁹. Use the hgRNA carrying Mouse for Actively Recording Cells 1 (MARC1) chimeric mouse with 60 distinct hgRNA loci in their genome⁷. In parallel, express Cas9-TdT fusion, attached by a T2A self-cleaving linker in primary neurons derived from the MARC1 line via transfection. For both these systems, next analyze the percentage of cells in which TdT based edits were recorded and calculate the percentage of each nucleotide incorporation to establish a “0” control condition. “0” is defined as a no input signal control.

Characterize TdT-Based Insertions Upon Altering Intracellular Nucleotide Pool:

Next, repeat the recording set-up with direct injection of either deoxyribonucleosides or other small molecules known to alter intracellular nucleotide pools (like dCF etc). This can be done with dAdo, dGuo, dThd, dCyt and dCF. This will establish the condition that gives the maximum output signal in neurons. Finally, establish how the dose response will look for the best signal recorded. Trying at least 10 different concentrations for that input signal. Characterizing the control conditions in detail will be very important for the final application of spatial reconstructions. For the next set of experiments, use the input signal(s) that resulted in the highest output and or best dose response.

Spatial Reconstruction in Mouse Brains:

Treat the neuronal population that was injected the RNP and TdT in, with the relevant input signal established in aim (2) (FIG. 2D). After a few hours of recording, collect the tissue samples and sequence the cells in situ^{9,11,20}. Preserving tissue samples in tissue sections can help supplement the spatial resolution even more. Since the concentration of the input signal will slowly diffuse over the depth of the tissue sample, sequencing the output signal at different locations will help us establish connectivity.

Spatial Reconstruction for Entire Mouse Brain:

Finally, an experiment may be performed in the entire mouse brain via several carefully planned input signal injections.

For this experiment, establish a mouse line with genomically integrated Cas9-TdT fusion (linked by a T2A linker) which will be crossed with the MARC1 mouse line (FIG. 1C). Trials with plasmid based Cas9-TdT fusion expression attempted in neuronal cells cultures in aim 1 will help with establishing the best way to genetically encode the Cas9-TdT here. Then, strategically inject the ideal input signal established in aim 2 and 3 to the mouse brain and record for several hours in different brain regions independently. Next, carry out in situ sequencing for each brain region individually and thus establish the entire connectome at high neuronal resolution.

### REFERENCES

1. Reina-Campos, M., Moscat, J. & Diaz-Meco, M. Metabolism shapes the tumor microenvironment. Curr. Opin. Cell Biol. 48, (2017).
2. Keller, L. & Pantel, K. Unravelling tumour heterogeneity by single-cell profiling of circulating tumour cells. Nat. Rev. Cancer 19, (2019).
3. Qi, M., Philip, M. C., Yang, N. & Sweedler, J. V. Single Cell Neurometabolomics. ACS Chem. Neurosci. 9, (2018).
4. Rubakhin, S. S., Romanova, E. V, Nemes, P. & Sweedler, J. V. Profiling metabolites and peptides in single cells. Nat. Methods 8, (2011).
5. Kalhor, R., Mali, P. & Church, G. M. Rapidly evolving homing CRISPR barcodes. Nat. Methods 14, 195-200 (2017).
6. Loveless, T. B. et al. Ordered insertional mutagenesis at a single genomic site enables lineage tracing and analog recording in mammalian cells. bioRxiv 639120 (2019). doi:10.1101/639120
7. Church, G. M. Developmental barcoding of whole mouse via homing CRISPR. 9804, (2018).
8. Zador, A. et al. Probing the connectivity of neural circuits at single-neuron resolution using high-throughput DNA sequencing. Nat. Preced. (2011). doi:10.1038/npre.2011.6452.1
9. Chen, X. et al. High-Throughput Mapping of Long-Range Neuronal Projection Using In Situ Sequencing. Cell 179, 772-786.e19 (2019).
10. Marblestone, A. H. et al. Conneconomics: The Economics of Large-Scale Neural Connectomics. bioRxiv 001214 (2013). doi:10.1101/001214
11. Marblestone, A. H. et al. Rosetta Brains: A Strategy for Molecularly-Annotated Connectomics. (2014).
12. Farzadfard, F. & Lu, T. K. Emerging applications for DNA writers and molecular recorders. Science 361, 870-875 (2018).
13. Sheth, R. U. & Wang, H. H. DNA-based memory devices for recording cellular events. Nat. Rev. Genet. 19, 718-732 (2018).
14. Bhan, N. J. et al. Recording temporal data onto DNA with minutes resolution. bioRxiv 634790 (2019). doi:10.1101/634790
15. Kalhor, R. et al. Developmental barcoding of whole mouse via homing CRISPR. Science (80-.). 361, eaat9804 (2018).
16. Ross, D. D., Akman, S. A., Schrecker, A. W. & Bachur, N. R. Effects of Deoxynucleosides on Cultured Human Leukemia Cell Growth and Deoxynucleotide Pools. Cancer Res. 41, (1981).
17. Gangi-peterson, L. et al. Nucleotide pool imbalance and adenosine deaminase deficiency induce alterations of N-region insertions during V (D) J recombination Find the latest version: Nucleotide pool imbalance and adenosine deaminase deficiency induce alterations of N-region i. (1999).
18. Cristalli, G. et al. Adenosine deaminase: Functional implications and different classes of inhibitors. Med. Res. Rev. 21, 105-128 (2001).
19. Staahl, B. T. et al. Efficient genome editing in the mouse brain by local delivery of engineered Cas9 ribonucleoprotein complexes. Nat. Biotechnol. 35, 431-434 (2017).
20. Lee, J. H. et al. Fluorescent in situ sequencing (FIS SEQ) of RNA for gene expression profiling in intact cells and tissues. (2015). doi:10.1038/nprot.2014.191

What is claimed is:

1. A DNA-synthesis based recording system, comprising a Cas, a homing guide RNA (hgRNA), and a terminal deoxynucleotidyl transferase (TdT), wherein the Cas, the hgRNA, and the TdT are all comprised within a single cell.

2. The DNA-synthesis based recording system of claim 1, wherein the Cas is Cas9.

3. The DNA-synthesis based recording system of claim 1, wherein the Cas9 forms a complex with hgRNA and targets a DNA locus of the hgRNA.

4. The DNA-synthesis based recording system of claim 3, wherein the hgRNA spacer sequence is diversified after each edit.

5. The DNA-synthesis based recording system of claim 1, wherein the TdT is directed to the double-stranded breaks created by Cas at the hgRNA sites, and the TdT adds at least one nucleotide at the double-stranded breaks, and wherein the at least one nucleotide optionally comprises a barcode.

6. The DNA-synthesis based recording system of claim 5, wherein the identity of the nucleotide added by TdT depends on the concentration of nucleotides in the cell.

7. The DNA-synthesis based recording system of claim 5, wherein the TdT-directed base additions can be altered by altering the nucleotide concentration.

8. The DNA-synthesis based recording system of claim 1, wherein a change in TdT-based nucleotide incorporation into a hgRNA double-stranded break is defined as an output signal.

9. The DNA-synthesis based recording system of claim 8, wherein the output signal is detectable with in situ sequencing.

10. The DNA-synthesis based recording system of claim 1, wherein the cell is a neuron.

11. The DNA-synthesis based recording system of claim 10, wherein the neuron is within the brain of a living mammal.

12. A method of establishing connections between cells, comprising exposing at least two cells that each comprise a DNA-synthesis based recording system according to claim 1 to an organic environment comprising deoxyribonucleotide triphosphates (dNTPs) and a variable, allowing the TdT to add dNTPs to a DNA substrate, and isolating the DNA substrate; wherein the dNTP content of the DNA substrate corresponds to the concentration of the variable in the organic environment.
